(19) United States
(12) Patent Application Publication
(10) Pub. No.: US 2013/0030412 A1
(43) Pub. Date: Jan. 31, 2013

(54) Method and Device for Mixing Substances

(71) Applicant: Glucago LLC, West Lafayette, IN (US)

(72) Inventors: Rush L. Bartlett, II, West Lafayette, IN (US); Arthur L. Chlebowski, Lafayette, IN (US); Peter M. Greco, Jr., Fishers, IN (US)

(73) Assignee: GLUCAGO LLC, West Lafayette, IN (US)

(21) Appl. No.: 13/630,307

(22) Filed: Sep. 28, 2012

Related U.S. Application Data
(63) Continuation of application No. PCT/US2011/030910, filed on Apr. 1, 2011.
(60) Provisional application No. 61/331,827, filed on May 6, 2010.
(60) Provisional application No. 61/320,270, filed on Apr. 1, 2010.

(51) Int. Cl. A61M 37/00

(52) U.S. Cl. 604/520; 604/87

(57) ABSTRACT

A method and device for mixing substances to form a mixture that can he administered to a patient are disclosed. Such a device can include a hollow cylindrical member, a barrier sealing an opening of the member, and a tether attached to the barrier so that the harrier at least partially ruptures when the tether is pulled in a direction away from the barrier. Such a method for mixing substances can include providing a device with a first substance contained within the hollow cylindrical member. The method can also include the step of inserting the device into the barrel of an administering system, where the administering system includes a second substance, and the step of pulling the component in a direction away from the barrier such that the tether causes the barrier to at least partially rupture.

### CROSS REFERENCE TO RELATED APPLICATIONS

This international patent application claims the benefit of and incorporates by reference herein the disclosures of U.S. Ser. No. 61/320,270, tiled Apr. 1, 2010, and U.S. Ser. No. 61/331,827, filed May 6, 2010.

### BACKGROUND

The medical industry often administers pharmaceuticals and other substances to patients through intradermal, intramuscular, intravenous, intranasal, or subcutaneous delivery using syringes, sprayers, and droppers. Such substances may include vaccines, proteins, peptides, chemicals, antibodies, antivenoms, conjugated groups, nanoparticles, anti-viral agents, drugs, sustained release vehicles, antibiotics, anti-toxins, biological warfare agent antidotes, chemical warfare agent antidotes, anesthesia medications, nausea medications, heart attack medications, combinations of various pharmaceuticals and substances, and many other agents. Many of the medical compounds used to form the substances listed above are stored in solid form because of the increase in shelf life and improved stability. These solid compounds must be mixed with a solution before being administered to the body.

There are several devices and methods currently available that allow medical personnel to mix and administer substances. However, these current devices and methods have serious drawbacks. For example, the current devices are difficult to manufacture and assemble. Also, the assemblies that separate substances in current devices are prone to leaks, which allow substances to prematurely mix with other substances. In some cases, the devices have a solid bottom and have a stopper placed in the opening to seal it. For such containers, the user typically has to manually mix two components and then add the mixture into a drug administration device. The current methods of mixing and administering substances are complex and require many user interaction steps of which some can be dangerous or cumbersome. Because many drugs are held in liquid form, the current methods typically require refrigeration or have decreased shelf life without refrigeration. As a result, there exists a need for improved devices and methods for mixing and administering substances in an effective and efficient manner.

### SUMMARY

The present disclosure discloses a method and device for mixing substances to form a mixture that can he administered to a patient.

In one embodiment, a device for mixing substances includes a hollow cylindrical member defining a first end and a second end, a barrier sealing the opening in the first end of the member, and a tether attached to the barrier such that when the tether is pulled in a direction away from the barrier, the barrier at least partially ruptures.

In another embodiment, a method of administering a substance to a patient includes providing a substance having a hollow cylindrical member defining a first end and a second end, a barrier that seals the opening in the first end of the member, and a tether attached to the barrier such that when the tether is pulled in a direction away from the barrier, the barrier at least partially ruptures. The method also includes the steps of providing a first substance contained within the hollow cylindrical member, pulling the tether in a direction away from the barrier causing the barrier to rupture, and administering at least a portion of the first substance to a patient.

In another embodiment, a device for facilitating mixing substances to form a mixture that can he administered to a patient includes a hollow cylindrical member defining a first end and a second end, a barrier that seals the opening in the first end of the member, and a component comprising a tool that is configured to seal the opening in the second end of the member and to be movable within the hollow cylindrical member allowing for the tool to break the seal of the barrier on the opening in the first end of the member.

In another embodiment, a method of administering a substance to a patient includes providing a device having a hollow cylindrical member defining a first end and a second end, a barrier that seals the opening in the first end of the member, and a component comprising a tool that is configured to seal the opening in the second end of the member and to be moveable within the hollow cylindrical member allowing for the tool to break the seal of the barrier on the opening in the first end of the member. The method also includes the steps of providing a first substance contained within the hollow cylindrical member, pushing the component in the direction of the barrier causing the tool to break the seal of the barrier on the opening in the first end of the member, and administering at least a portion of the first substance to a patient.

In another embodiment, a device for facilitating mixing substances to form a mixture that can be administered to a patient includes a hollow cylindrical member defining a first end and a second end, a barrier that seals the opening in the first end of the member, a component configured to seal the opening in the second end of the member, and a tether attached to the barrier and component such that when the component is pulled in a direction away from the barrier, the barrier at least partially ruptures.

In another embodiment, a method of mixing substances to form a mixture that can be administered to a patient includes providing a device having a hollow cylindrical member defining a first end and a second end, a barrier that seals the opening in the first end of the member, a component that seals the opening in the second end of the member, and a tether attached to the barrier and component such that when the component is pulled in a direction away from the barrier, the barrier at least partially ruptures. The method also includes the steps of providing a first substance contained within the hollow cylindrical member, inserting the device into an administering system, wherein the administering system includes a second substance, and pulling the component in a direction away from the barrier such that the tether causes the barrier to at least partially rupture and the first and second substances to mix.

In another embodiment, a device for facilitating mixing substances to form a mixture that can be administered to a patient includes a hollow cylindrical member defining a first end and a second end, a barrier that seals the opening in the first end of the member, and a component that is configured to seal the opening in the second end of the member, the component configured to break the seal of the barrier on the opening in the first end of the member when the component is pushed in the direction of the barrier within the hollow cylindrical member.

In another embodiment, a method of mixing substances to form a mixture that can be administered to a patient including providing a device comprising a hollow cylindrical member defining a first end and a second end, a barrier that seals the opening in the first end of the member, and a component that is configured to seal the opening in the second end of the member, the component configured to break the seal of the barrier on the opening in the first end of the member when the component is pushed in the direction of the barrier within the hollow cylindrical member. The method also includes the steps of providing a first substance contained within the hollow cylindrical member, inserting the device into an administering system, wherein the administering system includes a second substance, and pulling the component in a direction away from the barrier such that the tether causes the barrier to at least partially rupture and the first and second substances to mix.

### BRIEF DESCRIPTION OF THE DRAWINGS

The features and advantages of this disclosure, and the manner of attaining them, will be more apparent and better understood by reference to the accompanying drawings, wherein:

FIG. 1a shows a perspective view of a device according to at least one embodiment of the present disclosure.

FIG. 1b shows a perspective view of the device of FIG. 1a disposed within a barrel of a syringe.

FIG. 2a shows a perspective view of a device according to at least one embodiment of the present disclosure.

FIG. 2b shows a perspective view of the device of FIG. 2a disposed within a barrel of a syringe.

FIG. 3a shows a perspective view of a device according to at least one embodiment of the present disclosure.

FIG. 3b shows a perspective view of the device of FIG. 3a disposed within a barrel of a syringe.

FIG. 3c shows a perspective view of a device according to at least one embodiment of the present disclosure.

FIG. 3d shows a perspective view of a device according to at least one embodiment of the present disclosure.

FIG. 3e shows a perspective view of the device in FIG. 3d after the stick of the plunger has interacted with the barrier.

FIG. 3f shows a perspective view of a device according to at least one embodiment of the present disclosure.

FIG. 3g shows a perspective view of the device of FIG. 3f disposed within a barrel of a syringe.

FIG. 4 shows a perspective view of a device according to at least one embodiment of the present disclosure disposed within a barrel of a sprayer/dropper.

FIG. 5 shows a perspective view of a device according to at least one embodiment of the present disclosure disposed within a barrel of a syringe configured to be connected to an IV system.

FIGS. 6a, 7a, 8a show flowcharts of methods of using the devices in FIGS. 1a, 1b, 1c respectively.

FIGS. 6b, 7b, 8b, 9 show illustrations of sequences of the methods of FIGS. 6a, 7a, 8a, 8a respectively.

### DETAILED DESCRIPTION

For the purposes of promoting an understanding of the principles of the present disclosure, reference will now he made to the embodiments illustrated in the drawings, and specific language will be used to describe the same. It will nevertheless be understood that no limitation of the scope of this disclosure is thereby intended.

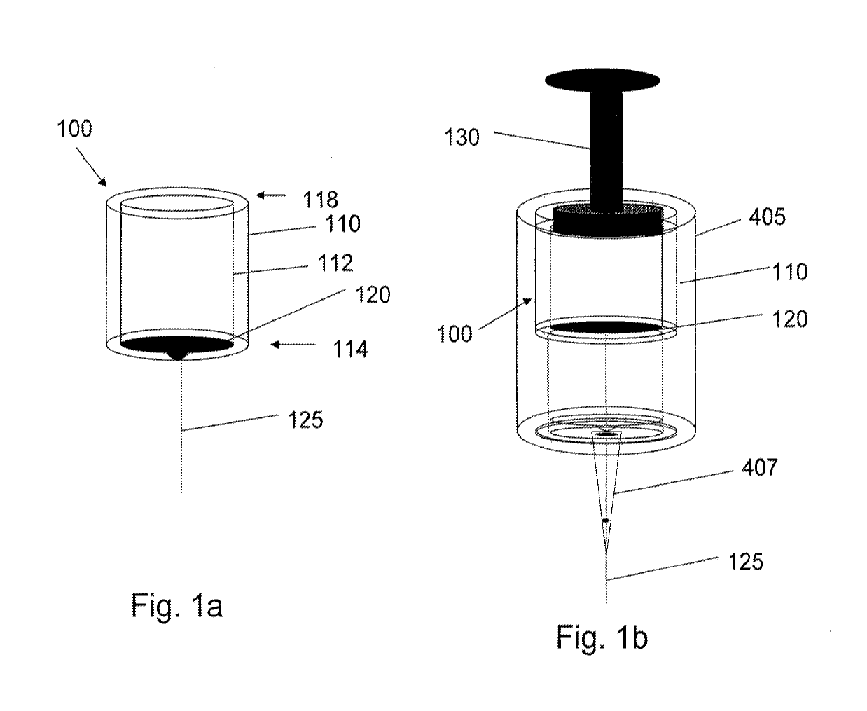

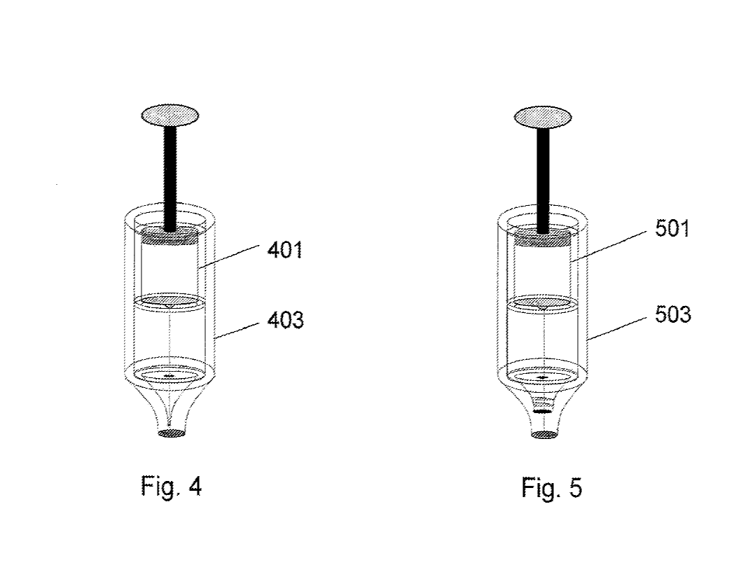

FIGS. 1a, 2a, 3a, 3c, 3d, and 3f illustrate medical units according to the present disclosure. The medical units can be used to facilitate mixing of substances to form a drug or vaccine, which can then be administered to a patient. As shown in FIGS. 1b, 2b, 3b, and 3g such medical units may be configured to be disposed within the barrel of a syringe. Of course, the medical units may be configured to be disposed within any type of administration device, such as, for example, sprayers (e.g., intranasal sprayers), droppers, luer-lok systems, and connectors to various devices, including intravenous connection systems and autoinjectors. For example, FIG. 4 shows a medical unit 401 within a sprayer/dropper device 403, and FIG. 5 shows a medical unit 501 within a syringe 503 that is configured to he connected to an intravenous system or other system. As further described below, the medical units of the present disclosure are configured to store one or more substances through the use of a plug or barrier portion. The units are configured to allow a user to rupture or dislodge the barrier portion so that the substance contained in the unit may be mixed with another substance in communication with the unit.

In FIG. 1a, a unit 100 is shown having a hollow cylindrical member 110. The member 110 has a hollow portion 112 and openings at its first end 114 and its second end 118. The hollow portion 112 of the member 110 is typically configured to be able to store various types of gas, liquid and solid substances. As shown in FIG. 1b, the member 110 may have an outer diameter that is substantially the same as the inner diameter of the barrel of a syringe 405 or other administering system. The member 110 may typically be formed of glass but may be formed of other materials. As shown in FIG. 1a, the unit 100 also includes a barrier 120 that seals the opening of the member 110 at the first end 114 or is at least partially disposed within the opening in the first end 114 such that the opening in the first end 114 is sealed. The barrier 120 may be formed of various rupturable materials including, but not limited to, foil, polymers, and the like. The barrier 120 may also be formed of various other materials, including, but not limited to, rubber.

As shown in FIG. 1a, the unit 100 also includes a tether 125 that is attached to the barrier 120. The barrier 120 can be ruptured or dislodged by pulling the tether 125 in a direction away from the barrier 120. With the barrier 120 ruptured or dislodged, a substance stored in the hollow portion 112 of the member 110 may escape from the unit 100 and mix with one or more substances in an administering system (e.g., syringe) or one or more substances otherwise in communication with the unit 100. As shown in FIG. 1b, when the unit 100 is disposed within, the barrel of a syringe, the tether 125 may be threaded through the needle 407 of the syringe 405. The tether 125 may be configured to substantially plug, block, or otherwise seal off the needle 407 such that a substance contained in the barrel of the syringe 405 is restricted from engaging the needle 407 while the tether 125 is disposed in the needle 407. In some circumstances, substances may be destabilized by interacting with the needle 407 or other metallic injection part. Therefore, by stopping substances from contacting the needle 407 or other metallic injection part, the tether 125 helps keep substances stable and in a condition to be effective to the recipient of the injection, spray, drop, and the like.

As shown in FIG. 1b. the unit 100 may optionally include a component 130 that is configured to seal the opening in the second end 118 of the member 110 and that optionally may be slidably received within at least a portion of the hollow portion 112. In FIG. 1b, the component 130 is a plunger that can be moved within the hollow portion 112. It should be noted that the component 130 may also be configured to move the unit 100 within the barrel of the syringe 405 to push any substances or mixtures located below the unit 100 within the barrel of the syringe 405 into the needle 407. For example, after the barrier 120 has been ruptured or dislodged and the substance in the unit 100 has mixed with a substance already in the barrel of the syringe 405, the unit 100 via forced applied to the component 130 may push the mixed substances through the needle 407.

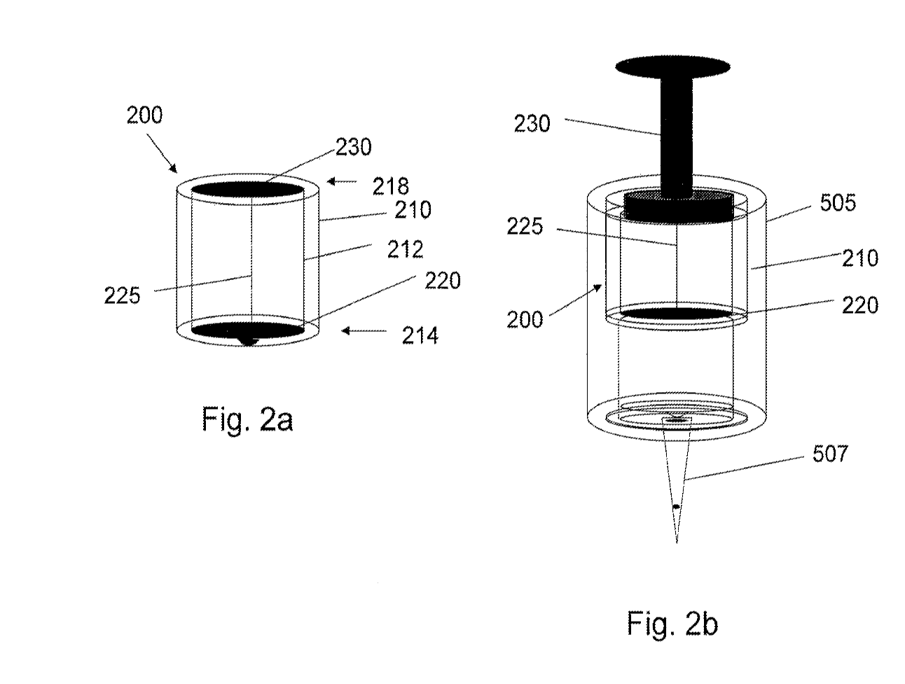

FIG. 2a shows a unit 200 that is similar to the unit 100 described above except the tether 225 is attached to both the component 230 and the barrier 220. FIG. 2b shows the unit 200 disposed within the barrel of a syringe 505. The barrier 220 can be ruptured or dislodged by pulling the component 230 in a direction away from the barrier 220. With the barrier 220 ruptured or dislodged, a substance stored in the hollow portion 212 of the member 210 may escape from the unit 200 and mix with one or more substances in the barrel of a syringe or one or more substances otherwise in communication with the unit 200.

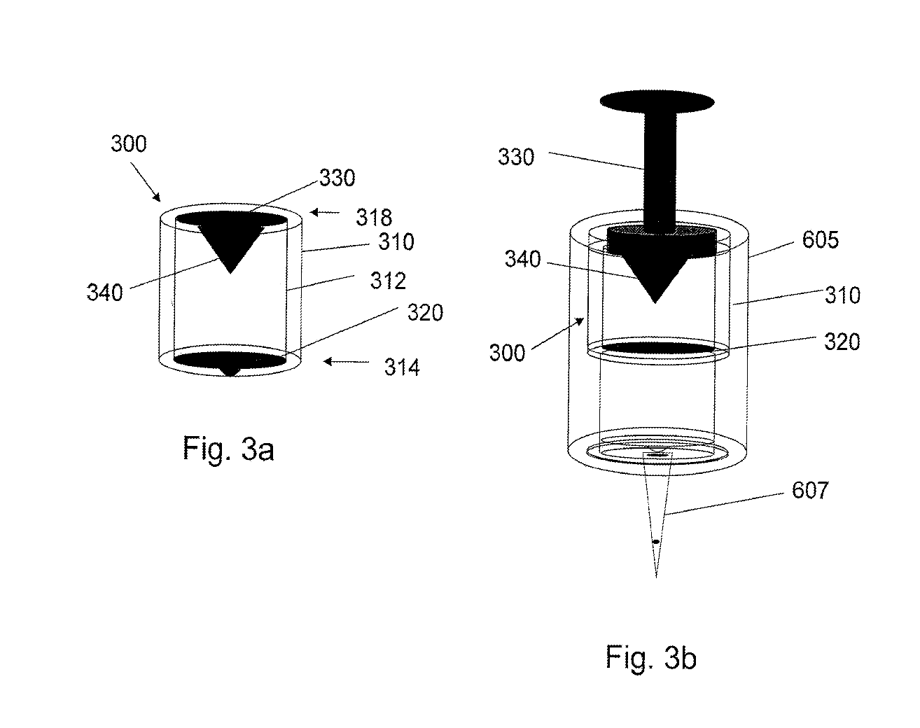

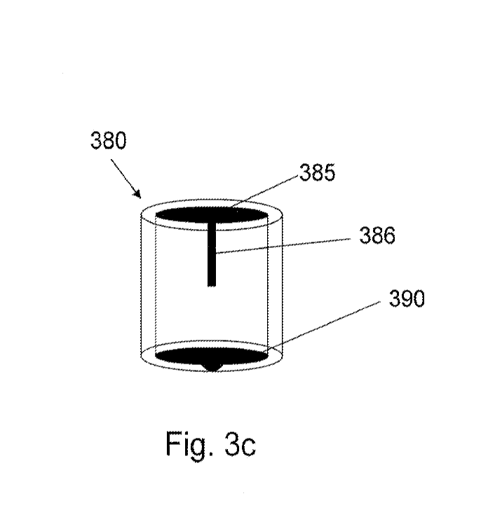

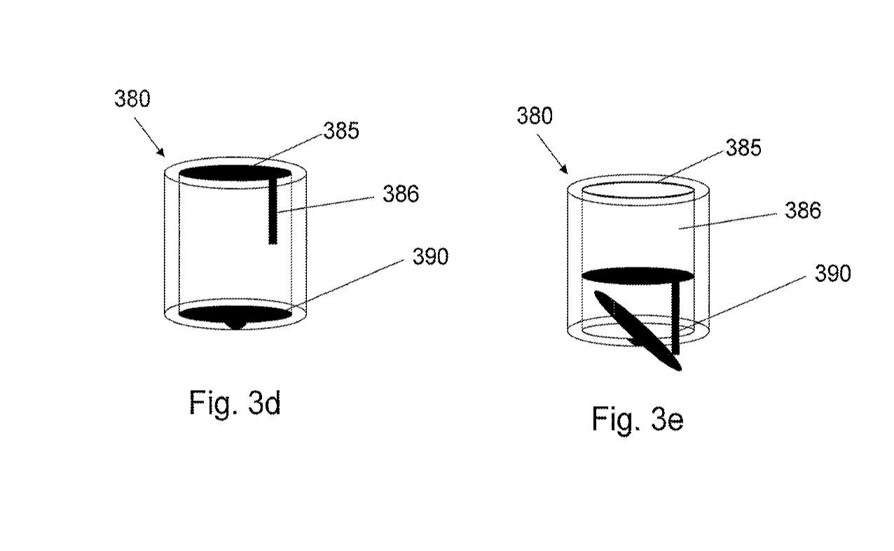

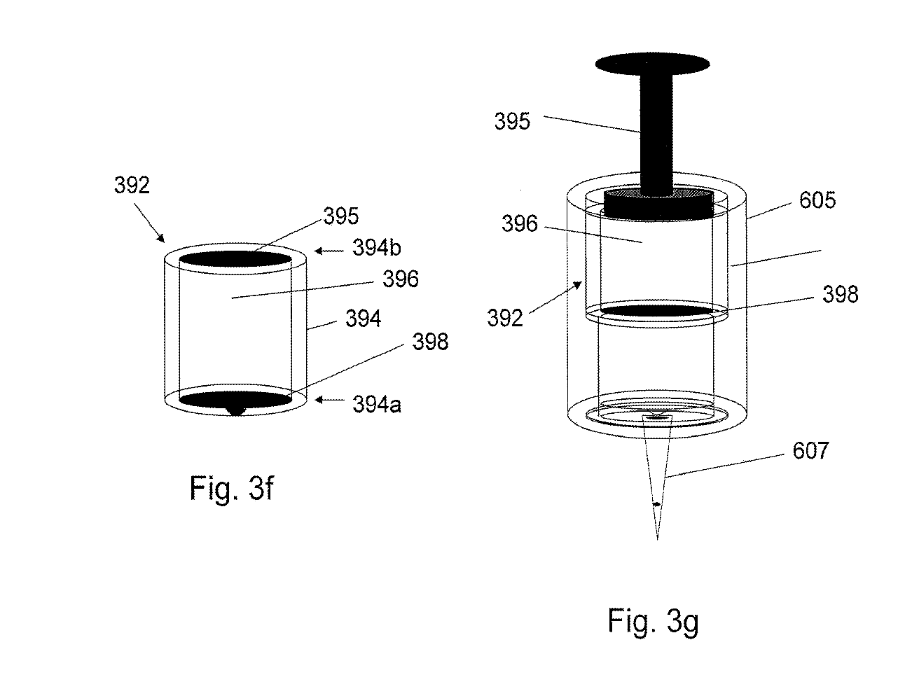

In FIG. 3a, a unit 300 is shown having a hollow cylindrical member 310. The member 310 has a hollow portion 312 and openings at its first end 314 and its second end 318. The hollow portion 312 of the member 310 is typically configured to be able to store various types of gas, liquid, and solid substances. As shown in FIG. 3b, the member 310 may have an outer diameter that is substantially the same as the inner diameter of the barrel of a syringe 605 or other administering system. The member 310 may typically be formed of glass but may be formed of other materials. The unit 300 also includes a barrier 320. As shown in FIG. 3a, the barrier 320 seals an opening in the member 310 at the first end 314 or is at least partially disposed within the opening in the first end 314 such that the opening in the first end 314 is sealed. The barrier 320 may he formed of various rupturable materials including, but not limit to, foil, polymers, and the like. The harrier 120 may also he formed of various other materials, including, but not limited to, rubber.

As shown in FIG. 3a, the unit 300 also includes a component 330 that is configured to seal the opening in the second end 318 of the member 310 and that is slidably received within at least a portion of the hollow portion 312. It should be noted that the component 330 may also be configured to move the unit 300 within the barrel of the syringe 605 to push any substances or mixtures within the barrel of the syringe 605 into the needle 607 or through some other administering device. In FIG. 3b, the component 330 is a plunger that can be moved within the hollow portion 312.

As shown in FIG. 3a, the component 330 includes a tool 340 that is configured to rupture or dislodge the harrier 320. In other words, the component 330 can be moved towards the harrier 320 so that the tool 340 engages the barrier 320 to dislodge or rupture the barrier 320. The tool 340 may be any type of instrument capable of rupturing or dislodging the barrier 320, including, but not limited to, a needle, a sharp polymer or glass object, a razor blade, a blunt instrument, and the like. With the harrier ruptured 320 or dislodged, a substance stored in the hollow portion 312 of the member 310 may be mixed with one or more substances in a syringe or one or more substances otherwise in communication with the unit 300.

For example, as shown in FIG. 3c, a unit 380 may include a component 385 having a stick 386 that is configured to dislodge the barrier 390. FIG. 3d shows the stick 386 that is off center. As shown in FIG. 3e, as the component 385 and stick 386 in FIG. 3d are pushed into contact with the barrier 390, the barrier 390 is dislodged. In a dislodged configuration, the barrier 390 allows for substances in the unit 380 to mix with substances in communication with the unit.

It should be noted that the barrier of a unit described, herein may be dislodged or ruptured by simply applying pressure (via gas, liquid, or solid) to the barrier or interacting with the barrier using the [symbol] . A plunger or the like may dislodge or rupture the barrier through increased gas pressure, pressure force from a liquid, pressure force by a solid substance, or by physical interaction between the plunger and barrier. An example of such a unit is shown in FIG. 3f. In FIG. 3f, a unit 392 is shown having a hollow cylindrical member 394. The member 394 has a hollow portion 396 and openings at its first end 394a and its second end 394b. The hollow portion 396 of the member 394 is typically configured to be able to store various types of gas, liquid, and solid substances. As shown in FIG. 3g, the member 394 may have an outer diameter that is substantially the same as the inner diameter of the barrel of a syringe 605. The member 394 may typically be formed of glass but may be formed of other materials. As shown in FIG. 3f, the unit 392 also includes a barrier 398 that seals an opening of the member 394 at the first end 394a or is at least partially disposed within the opening in the first end 394a such that the opening in the first end 394a is sealed. The barrier 398 may be formed of various rupturable materials including, but not limited to, foil, polymers, and the like. The barrier 398 may also he formed of various other materials, including, but not limited to, rubber.

As shown in FIG. 3f, the unit 392 also includes a component 395 that is configured to seal the opening in the second end 394b of the member 394 and that is movable within at least a portion of the hollow portion 396. In FIG. 3g, the component 395 is a plunger that can be moved within the hollow portion 396. It should he noted that the component 395 may also be configured to move the unit 392 within an administering system, such as the barrel of a syringe 605, to push any substances or mixtures within the administering system (e.g., within the barrel of the syringe 605 into the needle 607). For example, after the barrier 398 has been ruptured or dislodged and the substance in the unit 392 has mixed with a substance already in the barrel of the syringe 605, the unit 392 via force applied to the component 395 may push the mixed substances through the needle 607.

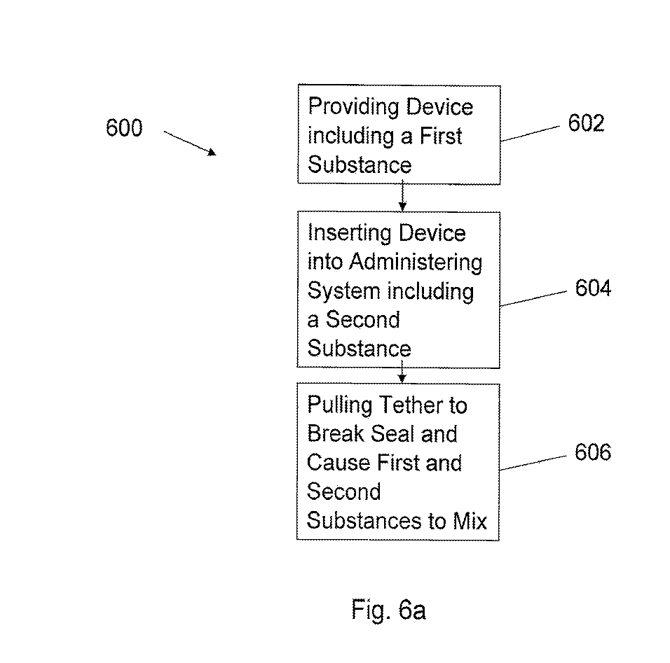

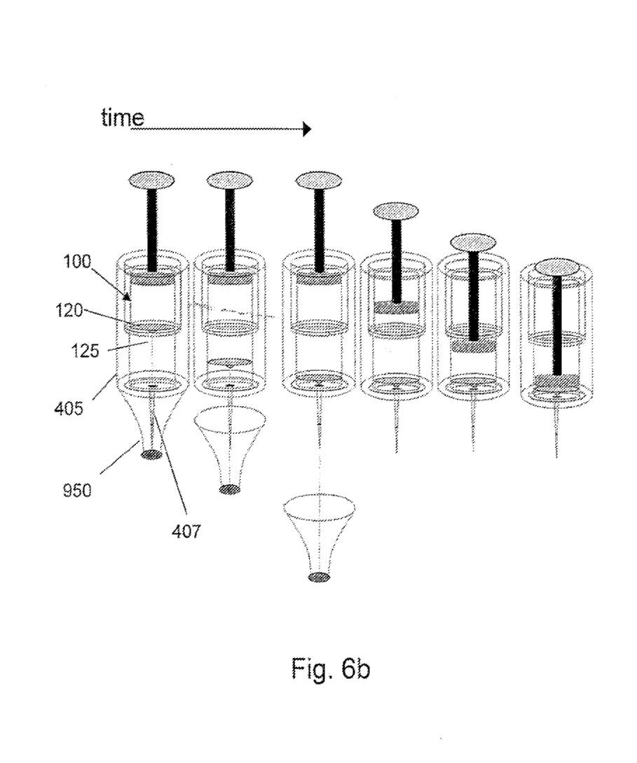

FIGS. 6a, 7a, and 8a illustrate methods of mixing substances to form a mixture that can be administered to a patient according to the present disclosure. The method 600 in FIG. 6a includes the step 602 of providing the medical unit 100 described above with a first substance contained, within the hollow portion of the hollow member. The method 600 also includes the step 604 of inserting the medical unit in at administering system, which contains a second substance. As shown in FIG. 1b, the medical unit may be inserted in a syringe such that the end of the unit with the barrier is closest, to the needle of the syringe. As mentioned previously, the medical unit may alternatively be inserted, into a sprayer, dropper, or other administering system. As shown in FIG. 6a, the method 600 also includes the step 606 of pulling the tether to cause the barrier to rupture or dislodge, resulting in the first and second substances mixing.

FIG. 6b is an illustration of the sequence of steps of using the unit 100 and syringe 405 of FIG. 1b according to at least one embodiment of method 600. In FIG. 6b, the tether 125 is initially coupled to a protective sleeve or cap 950 that fits over the needle 407 of the syringe 407. As shown in FIG. 6b, the sleeve 950 is then removed causing tension to be applied to the tether 125. With the tension on the tether 125, the barrier 120 is dislodged from the unit 100 and the tether 125 separates from the barrier 120. As noted above, the barrier 120 may rupture instead of being dislodged. It should be noted that the syringe 405 may include or operate alongside a system that is configured to pull on the tether 125 or remove the sleeve 950. The system may be initiated in various ways, such as, for example, a user pressing a button. The system may be spring-loaded or various other types of designs.

With the barrier 120 dislodged, a substance stored in the hollow portion 112 of the member 110 may escape from the unit 100 and mix with one or more substances stored in the barrel of the syringe. As shown in FIG. 6b, the plunger 130 is pushed downward toward the needle to eject the mixture comprising the mixed substances from the needle 407.

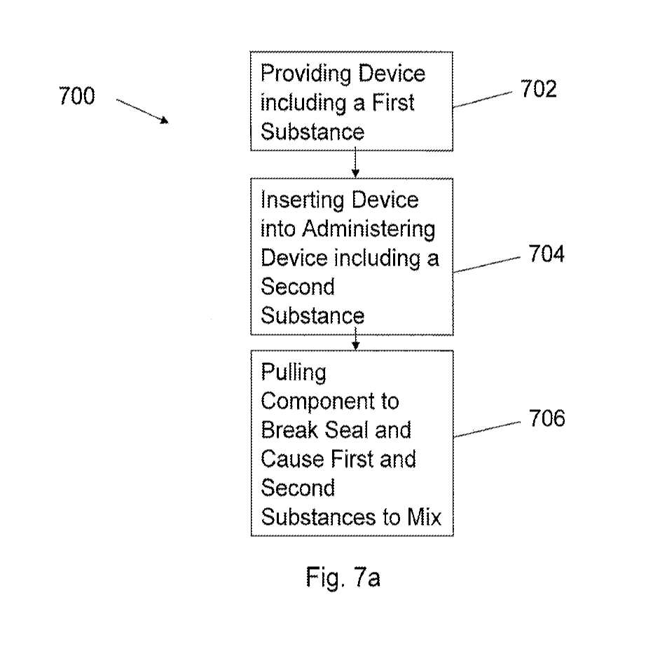

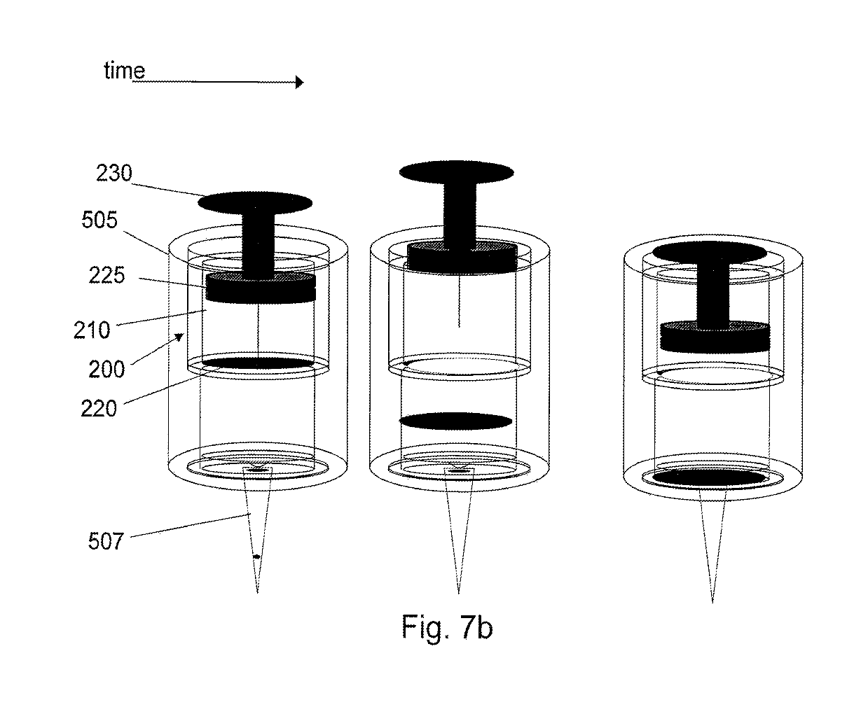

The method 700 in FIG. 7a includes the step 702 of providing the medical unit 200 described above with a first substance contained within the hollow portion of the hollow member. The method 700 also includes the step 704 of inserting the medical unit in an administering system, which contains a second substance. As shown in FIG. 2b, the medical unit may be inserted in a syringe such that the end of the unit with the barrier is closest to the needle of the syringe. As mentioned previously, the medical unit may alternatively be inserted into a sprayer, dropper, or other administering device. As shown in FIG. 7a, the method 700 also includes the step 706 of pulling the component to cause the tether to rupture or dislodge the harrier, resulting in the first and second substances mixing.

FIG. 7b is an illustration of the sequence of steps of using the unit 200 and syringe 505 of FIG. 2b according to at least one embodiment of method 700. As shown in FIG. 6b, the component or plunger 230 is pulled upwards causing the barrier 220 to be dislodged from the unit 200 and the tether 225 to separate from the barrier 220. As noted above, the barrier 220 may rupture instead of being dislodged. With the barrier 220 dislodged or ruptured, a substance stored in the hollow portion 212 of the member 210 may be mixed with one or more substances stored in the barrel of the syringe. As shown in FIG. 7b, the plunger 230 is pushed downward toward the needle to eject the mixture comprising the mixed substances from the needle 507.

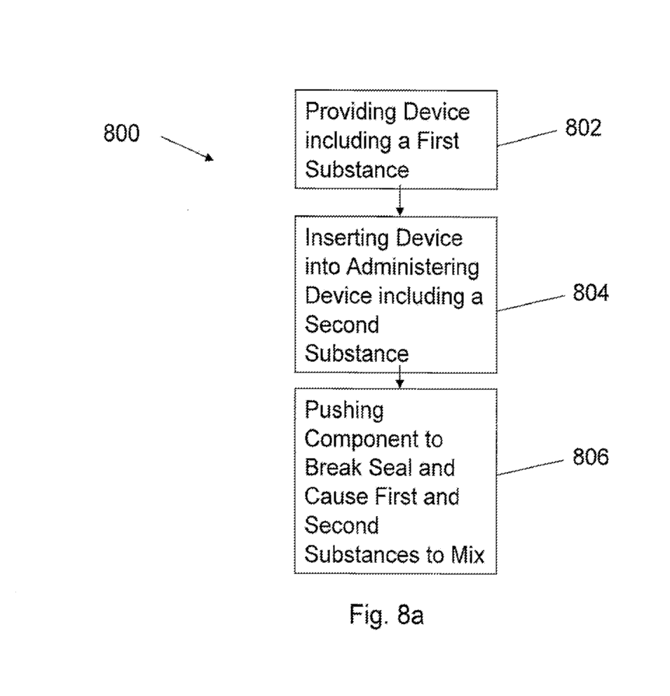

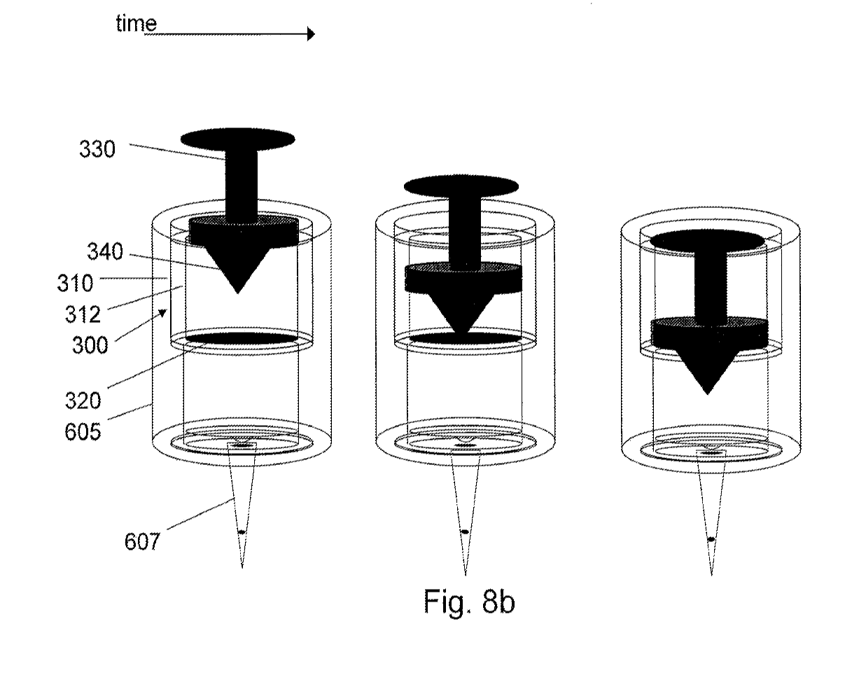

The method 800 in FIG. 8a includes the step 802 of providing the medical unit 300 described above with a first substance contained within the hollow portion of the hollow member. The method 800 also includes the step 804 of inserting the medical unit in an administering system, which contains a second substance. As shown in FIG. 3b, the medical unit may be inserted in a syringe such that the end of the unit with the barrier is closest to the needle of the syringe. As mentioned previously, the medical unit may alternatively be inserted into a sprayer, dropper, or other administering device. As shown in FIG. 8a, the method 800 also includes the step 806 of pushing the component to cause the tool to rupture or dislodge the barrier, resulting in the first and second substances mixing.

FIG. 8b is an illustration of the sequence of steps of using the unit 300 and syringe 605 of FIG. 3b according to at least one embodiment of method 800. As shown in FIG. 8b, the component or plunger 330 having the tool 340 is pushed towards the barrier 320, resulting in the barrier 320 being ruptured, or dislodged by the tool 340. With the barrier 320 ruptured or dislodged, a substance stored in the hollow portion 312 of the member 310 may be mixed with one or more substances stored in the barrel of the syringe. As shown in FIG. 8b, the plunger 330 is pushed downward toward the needle to eject the mixture comprising the mixed substances from the needle 607.

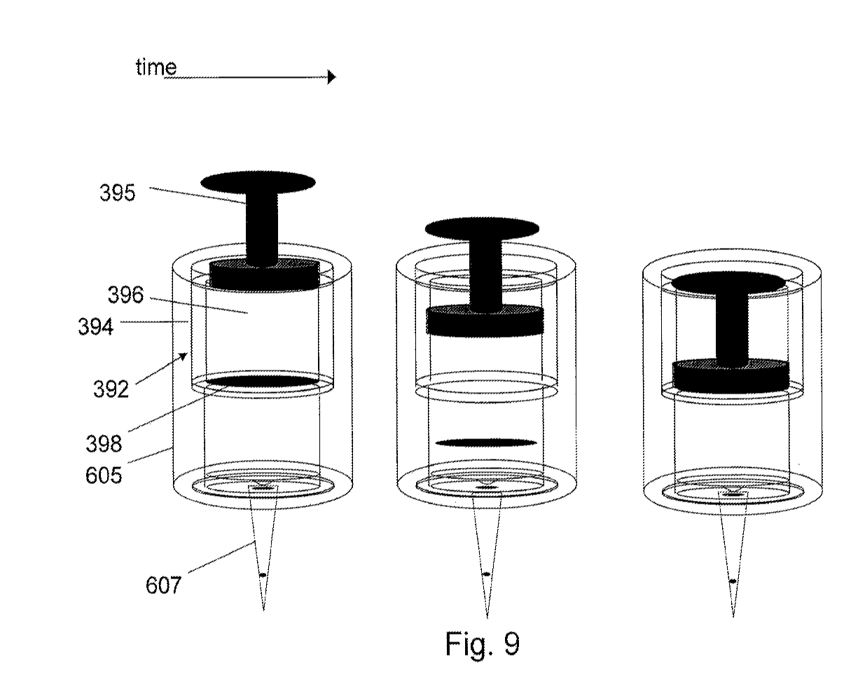

FIG. 9 is an illustration of the sequence of steps of using unit 392 and syringe 605 of FIG. 3g according to at least one embodiment of method 800. As shown in FIG. 9, the component or plunger 395, is pushed towards the barrier 398, resulting in the barrier 398 being ruptured or dislodged. With the barrier 398 ruptured or dislodged, a substance stored in the hollow portion 396 of the member 394 may be mixed with one or more substances stored in the barrel of the syringe 605. As shown in FIG. 9, the plunger 395 is pushed downward toward the needle 607 to eject the mixture comprising the mixed substances from the needle 607.

It should also be noted that one or more of the medical units described above may be placed in communication within a barrel of a single syringe or another medical device. In other words, two or more of the medical units described above may be placed in the barrel of a single syringe such that a user may rupture each of the respective barriers to mix the contents of all of the medical units. It should also be noted that each medical unit described herein may he configured to store any solids, liquids, or gases. The method and device described, above provides the capability of rapidly mixing substances to form a mixture with interaction by a user and administering the mixture to a patient.

The medical units described above may be used to mix lyophilized or spray dried substances such as, but not limited to, glucagon, epinephrine, heart attack rescue reagents, drugs, solid compounds, liquid compounds, vitamins, nucleic acids, proteins, peptides, vaccines, peptides, chemicals, antibodies, antivenoms, conjugated groups, nanoparticles, antiviral agents, drugs, sustained release vehicles, anesthesia medications, nausea medications, antibiotics, anti-toxins, biological warfare agent antidotes, chemical warfare agent antidotes, heart attack medications, combinations of the previous, and many other agents.

It should also be noted that the medical units described above can be fitted to a drug filling line to allow for depositing substances within the units. The method and device of the present disclosure eliminates operator error in mixing and checking the dosage of substances to be administered because the drug is not drawn into the drug delivery system from the container after a manual mixing.

While this disclosure has been described as having various embodiments, these embodiments according to the present disclosure can be further modified within the scope and spirit of this disclosure. This application is therefore intended to cover any variations, uses, or adaptations of the disclosure using its general principles. A practitioner may determine in a particular implementation that a plurality of components of the disclosed assembly may he combined in various ways, or that different components or different variations of the components may be employed to accomplish the same results. A practitioner may also determine in a particular implementation that a plurality of steps of the disclosed method may be combined in various ways, or that different steps or variations of the steps may be employed to accomplish the same results. Each such implementation falls within the scope of the present disclosure as disclosed herein and in the appended claims. Furthermore, this application is intended to cover such departures from the present disclosure as come within known or customary practice in the art to which this disclosure pertains.

That which is claimed is:

1. A device for facilitating the mixing of substances to form a mixture that can be administered to a patient, the device comprising:
a hollow cylindrical member defining a first end and a second end;
a barrier that seals the opening in the first end of the member; and
a tether attached to the barrier such that when the tether is pulled in a direction away from the barrier, the barrier at least partially ruptures.

2. The device of claim 1, further comprising a component configured to seal the opening in the second end of the member.

3. The device of claim 2, wherein the tether is attached to the component.

4. The device of claim 3, wherein the component comprises a plunger.

5. The device of claim 4, wherein the component comprises rubber.

6. The device of claim 1, further comprising a sleeve attached to the ether and configured to mate with the needle end of a syringe.

7. The device of claim 6, wherein the tether is removably attached to the barrier.

8. The device of claim 1, wherein the hollow cylindrical member comprises glass.

9. The device of claim 1, wherein the barrier comprises foil.

10. The device of claim 1, wherein the barrier comprises a polymer.

11. The device of claim 1, wherein the device is configured to be disposed within the barrel of a syringe.

12. A method of administering a substance to a patient, the method comprising:
providing a device comprising:
a hollow cylindrical member defining a first end and a second end;
a barrier that seals the opening in the first end of the member; and
a tether attached to the barrier such that when the tether is pulled in direction away from the barrier, the barrier at least partially ruptures;
providing a first substance contained within the hollow cylindrical member;
pulling the tether in a direction away from the barrier causing the barrier to rupture; and
administering at least a portion of the first substance to a patient.

13. A device for facilitating mixing substances to form a mixture that can be administered to a patient, the device comprising:
a hollow cylindrical member defining a first end and a second end;
a barrier that seals the opening in the first end of the member; and
a component comprising a tool, the component configured to seal the opening in the second end of the member and to be movable within the hollow cylindrical member allowing for the tool to break the seal of the barrier on the opening in the first end of the member.

14. The device of claim 13, wherein the tool comprises a polymer.

15. The device of claim 13, wherein the tool comprises glass.

16. The device of claim 13, wherein the component comprises a plunger.

17. The device of claim 16, wherein the component comprises rubber.

18. The device of claim 13, wherein the hollow cylindrical member comprises glass.

19. The device of claim 13, wherein the barrier comprises foil.

20. The device of claim 13, wherein the barrier comprises a polymer.

21. The device of claim 13, wherein the device is configured to be disposed within the barrel of a syringe.

22. A method of administering a substance to a patient, the method comprising:
providing a device comprising:
a hollow cylindrical member defining a first end and a second end;
a barrier that seals the opening in the first end of the member; and
a component comprising a tool, the component configured to seal the opening in the second end of the member and to be moveable within the hollow cylindrical member allowing for the tool to break the seal of the barrier on the opening in the first, end of the member;
providing a first substance contained within the hollow cylindrical member;
pushing the component in the direction of the barrier causing the tool to break the seal of the barrier on the opening in the first end of the member; and
administering at least a portion of the first substance to a patient.

23. A device for facilitating mixing substances to form a mixture that can be administered to a patient, the device comprising:
a hollow cylindrical member defining a first end and a second end;
a barrier that seals the opening in the first end of the member;
a component configured to seal the opening in the second end of the member; and
a tether attached to the barrier and component such that when the component is pulled in a direction away from the barrier, the barrier at least partially ruptures.

24. The device of claim 23, wherein the component comprises a plunger.

25. The device of claim 24, wherein the component comprises rubber.

26. The device of claim 23, wherein the hollow cylindrical member comprises glass.

27. The device of claim 23, wherein the barrier comprises foil.

28. The device of claim 23, wherein the barrier comprises a polymer.

29. The device of claim 23, wherein the device is configured to be disposed within the barrel of a syringe.

30. A method of mixing substances to form a mixture that can be administered to a patient, the method comprising:
providing a device comprising:
a hollow cylindrical member defining a first end and a second end;
a barrier that seals the opening in the first end of the member;
a component that is configured to seal the opening in the second end of the member; and
a tether attached to the barrier and component such that when the component is pulled in a direction away from the barrier, the barrier at least partially ruptures;
providing a first substance contained within the hollow cylindrical member;
inserting the device into an administering system, wherein the administering system includes a second substance; and
pulling the component in a direction away from the barrier such that the tether causes the barrier to at least partially rupture and the first and second substances to mix.

31. The method of claim 30, wherein the administering system is a syringe.

32. The method of claim 30, wherein the administering system is a sprayer.

33. The method of claim 30, wherein the hollow cylindrical member comprises glass

34. The method of claim 30, wherein, the barrier comprises foil.

35. The method of claim 30, wherein the barrier comprises a polymer.

36. A device for facilitating mixing substances to form a mixture that can be administered to a patient, the device comprising:
a hollow cylindrical member defining a first end and a second end;
a barrier that seals the opening in the first end of the member; and
a component that is configured to seal the opening in the second end of the member, the component configured to break the seal of the barrier on the opening in the first end of the member when the component is pushed in the direction of the barrier within the hollow cylindrical member.

37. The device of claim 36, wherein the component comprises a plunger.

38. The device of claim 37, wherein the component comprises rubber.

39. The device of claim 36, wherein the hollow cylindrical member comprises glass.

40. The device of claim 36, wherein the barrier comprises foil.

41. The device of claim 36, wherein the harrier comprises a polymer.

42. The device of claim 36, wherein the device is configured to be disposed within the barrel of a syringe.

43. A method of mixing substances to form a mixture that can be administered to a patient, the method comprising:
providing a device comprising:
a hollow cylindrical member defining a first end and a second end;
a barrier that seals the opening in the first end of the member; and
a component that is configured to seal the opening in the second end of the member, the component configured to break the seal of the barrier on the opening in the first end of the member when the component is pushed in the direction of the barrier within the hollow cylindrical member;
providing a first substance contained within the hollow cylindrical member;
inserting the device into an administering system, wherein the administering system includes a second substance; and
pulling the component in a direction away from the barrier such that the tether causes the barrier to at least partially rupture and the first and second substances to mix.
